(19) United States
(12) Patent Application Publication
(10) Pub. No.: US 2023/0225671 A1
(43) Pub. Date: Jul. 20, 2023

(54) WEARABLE HEALTH APPARATUS FOR THE COLLECTION OF WELLNESS DATA AND PROVIDING FEEDBACK THEREFROM TO THE WEARER

(71) Applicant: Circular, Paris (FR)

(72) Inventors: Amaury Kosman, Orsay (FR); Adiasa Suharno, Rodez (FR); Laurent Bsalis, Paris (FR)

(21) Appl. No.: 18/123,456

(22) Filed: Mar. 20, 2023

Related U.S. Application Data
(63) Continuation-in-part of application No. 16/797,038, filed on Feb. 21, 2020.

(51) Int. Cl. A61B 5/00

(52) U.S. Cl. CPC A61B 5/6802; A61B 5/6826; A61B 5/002; A61B 5/746; A61B 2562/166

(57) ABSTRACT

A health and wellness tracking ring that can be worn by a user at all times, that is unobtrusive and that collects health and wellness data about the user providing a means for the wearer to access and utilize the collected data for continuous monitoring and improvement of the wearer's health and wellness. The ring includes a control mechanical button that is easily accessible to the user while wearing. The ring includes replaceable outer shells and includes hardware and software that allow the user to communicate with software in a computer device such as a cell phone, cloud provider, table, personal computer or AI assistant.

### REFERENCE TO PRIOR APPLICATION

This application is a continuation in part of U.S. patent application Ser. No. 16/797,038 filed on Feb. 21, 2020, entitled WEARABLE HEALTH APPARATUS FOR THE COLLECTION OF WELLNESS DATA AND PROVIDING FEEDBACK THEREFROM TO THE WEARER AND METHOD OF USE THEREOF by Amaury Kosman, Adiasa Suharno and Laurent Bsalis.

### FIELD OF THE INVENTION

The field of this invention relates generally to the field of wearable health and fitness devices and more particularly toward wellness and smart rings and method of use thereof which is a ring that can be worn by a user at all times, that is unobtrusive and that collects health and wellness data about the user providing a means for the wearer to access and utilize the collected data for continuous monitoring and improvement of the wearer's health and wellness.

### DESCRIPTION OF THE PRIOR ART

Tracking of health and fitness information through the use of wearable data collection devices is known in the art in the form of devices that can wrap around a wearer's wrist or finger or other part of the body for the collection of data that the device can be programmed to receive, such as vital signs and the measurement of activities, such as steps walked or ran. It has been found that data collected through finger data is more accurate than data collected through the wrist.

The problem with existing devices is primarily the fact that they are not worn at all times, leaving gaps in the data collection when the user is not wearing the device. As a result, the conclusions drawn from the data are poor and therefore not useful to the user. The user needs to be wearing the data collection device while asleep as well as while awake, at all sorts of occasions from casual to elegant. Prior art devices fall short of providing the means for the meaningful collection of data.

It is the object of the instant invention to provide a wearable data collection device that overcomes the shortcomings of the prior art.

### SUMMARY OF THE INVENTION

The device of the instant invention provides for a wearable ring that acts as a computing device that can collect data from the wearer when wearing the ring. The ring includes a main body part that is molded and made of a nonconductive rigid polymer material on the outer surface. The inner surface of the ring, which contacts the skin on the finger of the wearer, is made of hypoallergenic polymer material and includes a raised portion on the inner surface therebetween creating a circular cavity through the inner surface with a flat part above which the raised portion is situated. Under the raised protrusion on the flat part is contained rigid-flex printed circuit board (PCB), the rigid-flex printed circuit board (PCB) comprising one or more of an infrared, red and green heart photoplethysmography sensor, an multi-axis accelerometer, a temperature sensor, electrodermal activity sensor (EDA), vibration motor, a mechanical button, flash memory, an RF Transceiver IC which has near field communication capability, power management integrated circuit, a curved rechargeable battery and contact pins or wireless charging technology for charging. The device includes external and interchangeable outer shells made of titanium or other metallic or nonmetallic materials and a coating.

The instant invention provides a wearable health tracker that is worn 24 hours a day and is therefore collecting more and relevant data to make personal recommendations. The tracker is a ring that can be worn on the finger of the user and can therefore be collecting data at all times of the day or night.

The ring has an inner layer and an outer layer that is customizable and detachable. The inner layer contains the electronics that can communicate with smartphones and mobile devices, personal computers, cloud provider and AI assistants. The ring is programmable to collect data such as predictions of the user's energy level and recovery time, detection of long-term lifestyle trends of the user, provide day and night clinical data including sleep patterns, predict a weakening immune system and the onset of illness and silently wake the user with full energy in the morning.

The interchangeable outer shell that includes RF components with NFC capabilities and other electronics can provide a variety of materials with which it is made, including durability, water resistance and aesthetic choices such as color. The inner ring includes the programmable electronics and the control in the form of buttons that extend upward through the outer ring.

The basic embodiment of the present invention teaches a wearable, wellness-tracking ring comprising: a main body portion of a fixed size made of a nonconductive rigid material wherein said main body portion has an outer surface, said outer surface being circular in shape and wherein said outer surface of said main body portion contains a central groove created by a first circular flange and a second circular flange, surrounding a perimeter of only said outer surface of said main body portion on either side of said outer surface; an inner surface of said main body portion opposing said outer surface wherein an aperture is created therethrough that is only partially circular in shape through which a finger of a user can fit therebetween and wherein said inner surface further comprises a raised portion that creates a non-circular profile for said aperture wherein said raised portion creates a flat plateau with smooth edges that is covered by a protective top and is made of a same material as said inner surface and containing no contact sensors but may still contain electronics wherein said protective top covers one or more housed electronic components that are programmed and protected thereunder to capture and communicate data from said user wherein said flat portion, when worn, is situated near the bottom of said user's finger proximate said user's palm so that said control button is oriented toward the thumb of said user for easy access while worn; a removable decorative, circular external outer shell affixable and detachable in said groove of said outer surface said removable shell having no cavities or grooves thereon thereby providing a smooth appearance; and a control button on said outer surface that is accessible to said user while said user's finger is placed through said aperture and said flat plateau is contacting skin on said user's finger and said button is reachable by said user.

The above embodiment can be further modified by defining that said main body portion and said outer shell are manufactured using an over-molding process.

The above embodiment can be further modified by defining that said one or more_electronic components are taken from the following: rigid-flex printed circuit board (PCB), the rigid-flex printed circuit board (PCB) comprising one or more of an infrared, red and green heart photoplethysmography sensor, an multi-axis accelerometer, a temperature sensor, electrodermal activity sensor (EDA), vibration motor, a mechanical button, flash memory, an RF Transceiver IC which has near field communication capability, power management integrated circuit, a curved rechargeable battery and contact pins or wireless charging technology for charging.

The above embodiment can be further modified by defining that an RF transceiver IC with near field communication capability is situated on said main body portion proximate said control button.

The above embodiment can be further modified by defining that electronic components are assembled as follows: the button, made of polymer and outer shell, made of polymer are assembled with an over-molding process thereby creating an outer part of the ring that has no cavity; electronics are glued to the outer part of the ring, i.e., the outer shell and button with automatic tooling; liquid polymer is injected into a mold that contains the outer part and the electronics to create the inner part that is in contact with the finger to form a single indivisible part wherein the mold forms a cavity for the inner part of the ring to embody the electronic glued on the outer-shell part.

The above embodiment can be further modified by defining that said data that is collected is communicated to a device, such as a cell phone, a tablet, a personal computer, cloud provider or an AI assistant and performs no execution software function.

The above embodiment can be further modified by defining that software is installed in a cloud provider and/or said cell phone, tablet, personal computer or, cloud provider or AI assistant such that said software can utilize said collected and communicated data to provide feedback to said user wherein said feedback includes one or more of the elements taken from the following: making action recommendations for better health and wellness; provide alerts through said vibration motor such as alarms, reminders or alerting said user when said ring has traveled too far from said cell phone, tablet, personal computer, cloud provider or AI assistant.

An alternate embodiment of the instant invention teaches a method of collecting personal health data and providing continuous feedback thereof to a user comprising the steps of: acquiring a wearable, wellness-tracking ring, said ring further comprising: a main body portion of a fixed size made of a nonconductive rigid material wherein said main body portion has an outer surface, said outer surface being circular in shape and wherein said outer surface of said main body portion contains a central groove created by a first circular flange and a second circular flange, surrounding a perimeter of said outer surface of said main body portion on either side of said outer surface; an inner surface of said main body portion opposing said outer surface wherein an aperture is created therethrough that is only partially circular in shape through which a finger of a user can fit therebetween and wherein said inner surface further comprises a raised portion that creates a non-circular profile for said aperture wherein said raised portion creates a flat plateau with smooth edges that is covered by a protective top and is made of a same material as said inner surface and containing no contact sensors wherein said protective top covers one or more housed electronic components that are programmed and protected thereunder to capture and communicate data from said user wherein said flat portion, when worn, is situated near the bottom of said user's finger proximate said user's palm so that said control button is oriented toward the thumb of said user for easy access while worn; a removable decorative, circular external outer shell affixable and detachable in said groove of said outer surface said removable shell having no cavities or grooves thereon thereby providing a smooth appearance; and a control button on said outer surface that is accessible to said user while said user's finger is placed through said aperture and said flat plateau is contacting skin on said user's finger and said button is reachable by said user; inserting a user's finger into said aperture; pairing said ring to said user's personal external computing device; wearing said ring continuously such that continuous data is communicating said external computing device; and collecting said continuous data into said external computing device for use by said user.

The above embodiment can be further modified by defining that said main body portion and said outer shell are manufactured using an over-molding process.

The above embodiment can be further modified by defining that said one or more_electronic components are taken from the following: rigid-flex printed circuit board (PCB), the rigid-flex printed circuit board (PCB) comprising one or more of an infrared, red and green heart photoplethysmography sensor, an multi-axis accelerometer, a temperature sensor, electrodermal activity sensor (EDA), vibration motor, a mechanical button, flash memory, an RF Transceiver IC which has near field communication capability, power management integrated circuit, a curved rechargeable battery and contact pins or wireless charging technology for charging.

The above embodiment can be further modified by defining that an RF transceiver IC with near field communication capability is situated on said main body portion proximate said control button.

The above embodiment can be further modified by defining that electronic components are assembled as follows: the button, made of polymer and outer shell, made of polymer are assembled with an over-molding process thereby creating an outer part of the ring that has no cavity; electronics are glued to the outer part of the ring, i.e., the outer shell and button with automatic tooling; liquid polymer is injected into a mold that contains the outer part and the electronics to create the inner part that is in contact with the finger to form a single indivisible part wherein the mold forms a cavity for the inner part of the ring to embody the electronic glued on the outer-shell part.

The above embodiment can be further modified by defining that said data that is collected is communicated to a device, such as a cellphone, a tablet, a personal computer a cloud provider, or an AI assistant and performs no execution software function.

The above embodiment can be further modified by defining that software is installed in said cell phone, tablet, personal computer, cloud provider or AI assistant such that said software can utilize said collected and communicated data to provide feedback to said user wherein said feedback includes one or more of the elements taken from the following: making action recommendations for better health and wellness; provide alerts through said vibration motor such as alarms, reminders or alerting said user when said ring has traveled too far from said cell phone, tablet, personal computer, cloud provider or AI assistant.

The above embodiment can be further modified by defining that said control button is utilized as an emergency signaler to said cell phone, tablet, personal computer, cloud provider or AI assistant.

The above embodiment can be further modified by defining that said ring and associated software are programmed to induce sleep, give indications, interact with the user and evoke relaxation through pace breathing and haptic feedback.

### BRIEF DESCRIPTION OF THE DRAWINGS

For a better understanding of the present invention, reference is to be made to the accompanying drawings. It is to be understood that the present invention is not limited to the precise arrangement shown in the drawings.

FIG. 1 is an isometric view of the assembled ring of the instant invention.

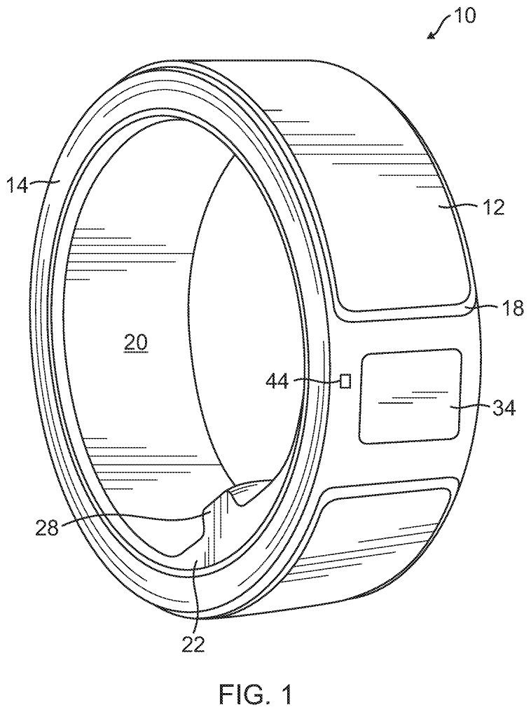

FIG. 2 is an exploded view of an illustrative embodiment of the ring of the instant invention showing the main body ring and two outer shells that attach thereto.

FIG. 3 is an isometric view of an illustrative outer shell for the ring of the instant invention.

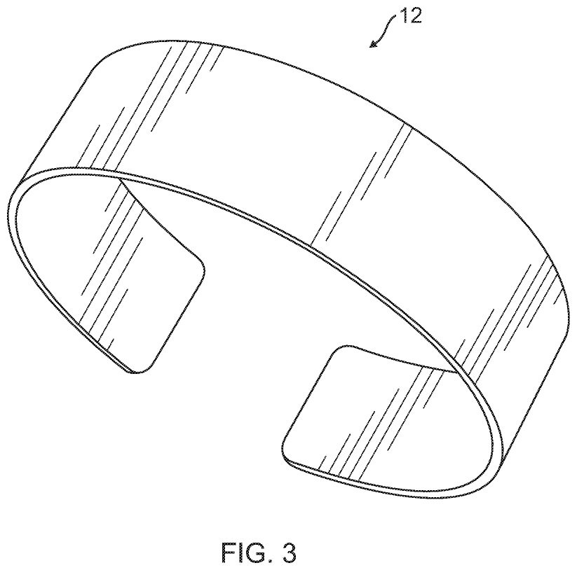

FIG. 4 is a close-up view of the heart photoplethysmography sensor in the raised portion on the inner surface of the main body of the ring of the instant invention.

FIG. 5 is an illustrative view of the ring of the instant invention around the finger of a user showing how the user can access the power button on the outer surface of the main body of the ring.

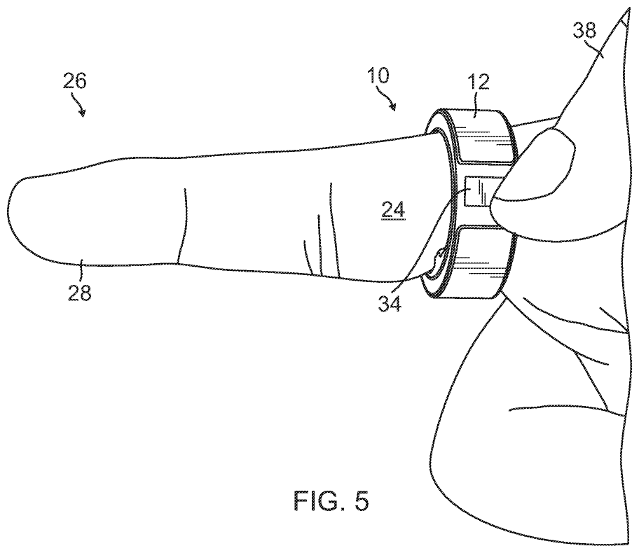

FIG. 6 is a view illustrating how the ring can charge on a power dock.

FIG. 7A is an isometric view of the ring of the instant invention showing the flat portion of the ring.

FIG. 7B is a side view of the ring of the instant invention showing the flat portion of the ring.

FIG. 8 is a schematic diagram outlining the electrical components contained in the flat portion of the ring of the instant invention.

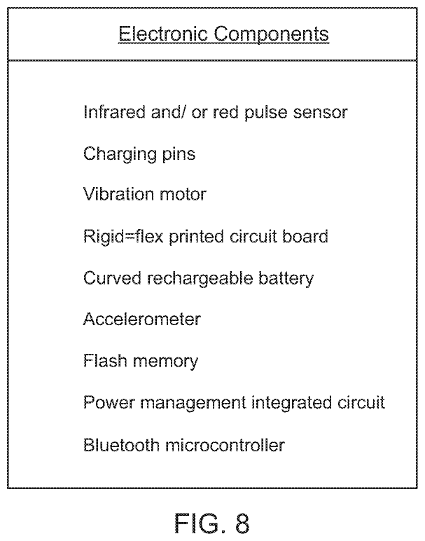

FIG. 9 is a table outlining some of the software functions included in communication with the ring of the instant invention.

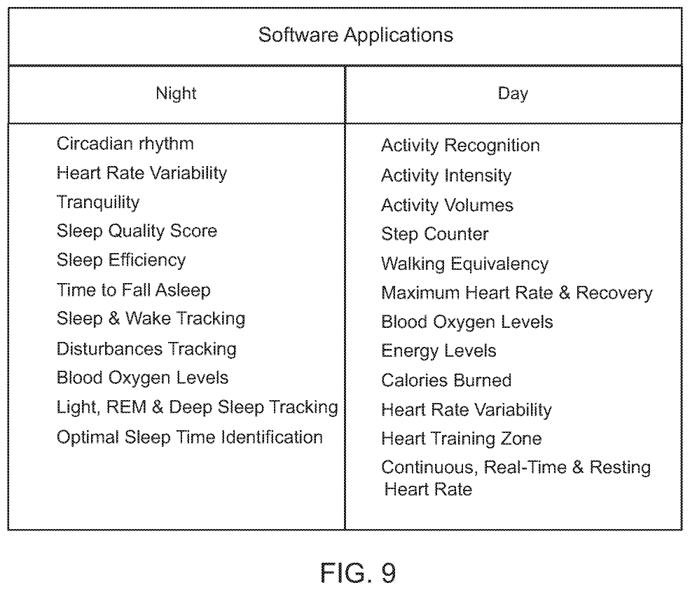

FIG. 10 is flow chart showing the manufacturing process of the ring of the instant invention.

### DETAILED DESCRIPTION OF A PREFERRED EMBODIMENT

Turning to the drawings, the preferred embodiment is illustrated and described by reference characters that denote similar elements throughout the several views of the instant invention.

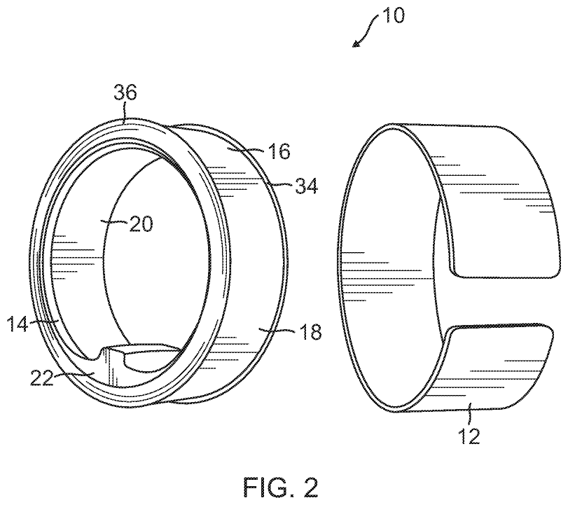

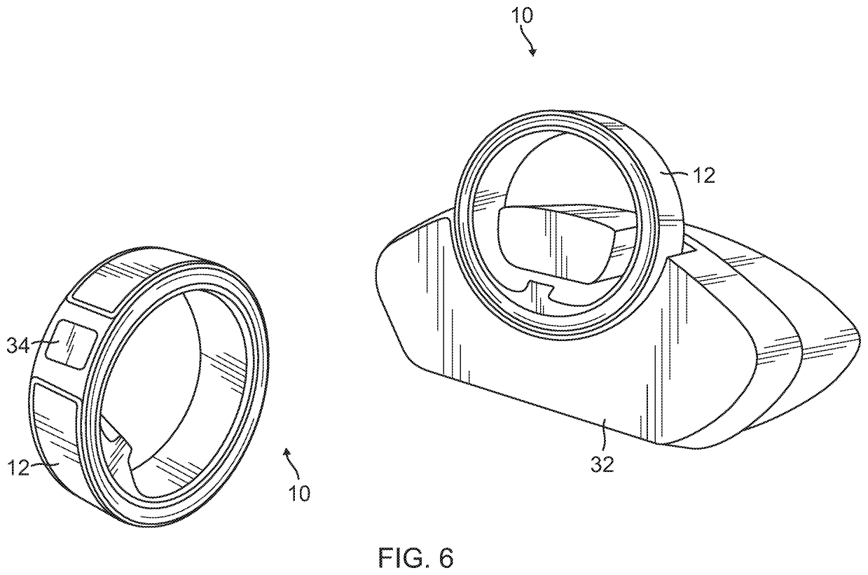

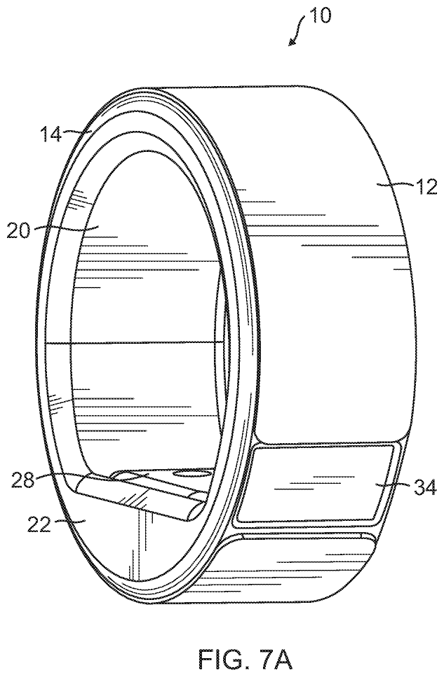

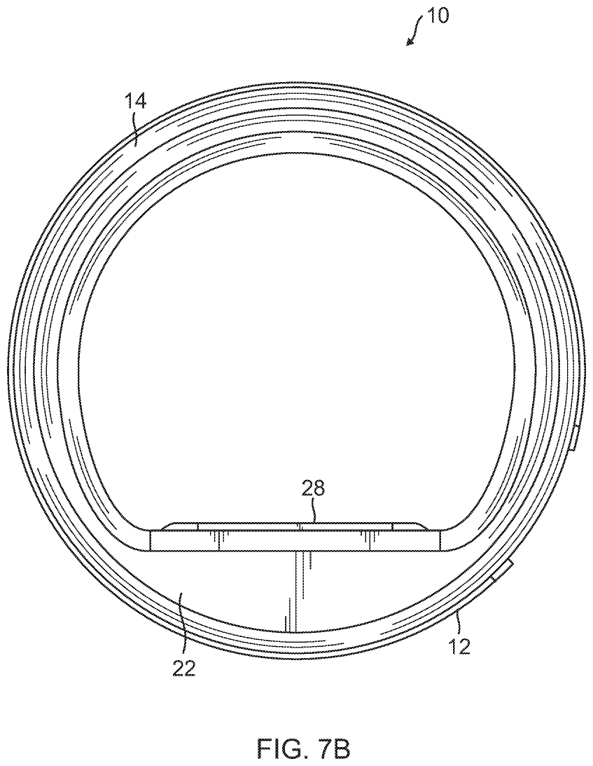

The ring 10 of the instant invention has two main parts with each ring for separate ring sizes being manufactured separately, i.e., the individual rings are inelastic and not sizable. This is because the ring 10 is made of a nonconductive rigid material. The first main part is the changeable outer shell 12 that has a smooth continuous surface without cavities or grooves, and the second part is the main body 14. The main body 14 breaks down into smaller parts. Viewing the ring 10 from the outside in (See FIG. 2), first is the outer shell 12, which is made out of rigid material. The outer shell 12 serves as a protective outer portion to the assembled ring 10. The main body 14 includes a groove 16 on the outer surface 18 that provides a slot for insertion of the changeable outer shell 12. The main body 14 also has an inner surface 20 that contacts the skin 24 on the finger 28 of the wearer 26. The inner surface 20 is substantially circular around most of the inner circumference of the ring 10, but bottoms out in a flat portion 22 (See FIGS. 7A-7B). The flat portion 22 provides a platform into which the electronics including a vibration motor and flash memory and the assembled PCB are housed, covered with an overmold housing 28. This flat portion 22 platform also includes charging pins 30 that allow the ring 10 to be charged on a charging dock 32 (See FIG. 6.) It is important to note that this flat portion 22 is not a sensor, but merely a cover for the electronics house thereunder.

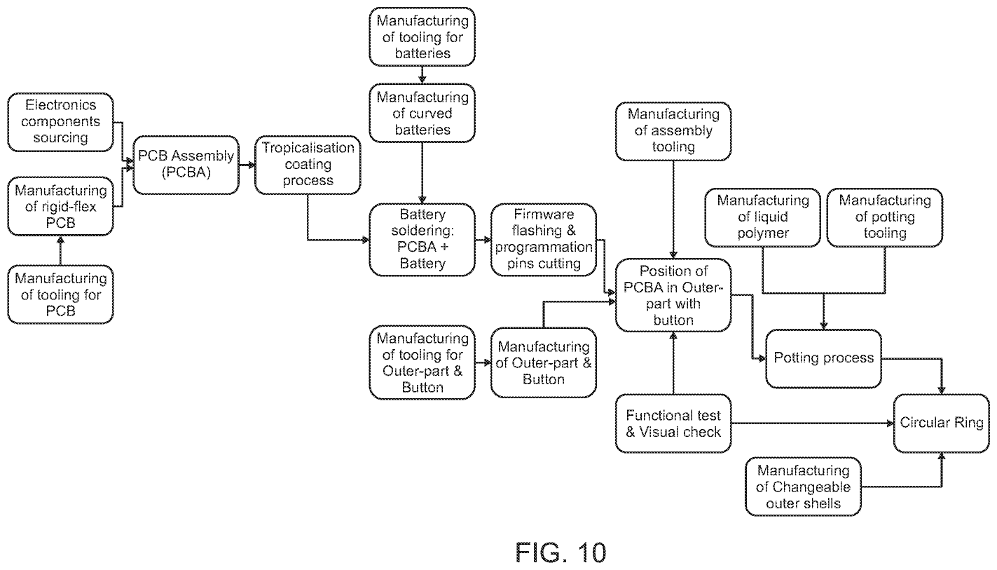

The ring 10 is made through a specific over molding process which are elaborated on in FIG. 10. The use of rigid plastic as the material of the ring 10 is significant because it allows the device 10 to conduct the temperature of the electronics inside the main body 14 and eliminates the possibility of falsifying the skin temperature reading of the user.

The ring 10 of the instant invention includes a button 34. This button 34 provides control the user and can be used to stop vibrations, to control other devices and to trigger any other actions programmed into the electronics.

One feature of the instant invention is the customization achievable through the detachable outer shells 12. This allows the user to change the look as desired by the user. The outer shells 14 are preferably made of titanium or other metallic or nonmetallic materials and a coating of a variety of different colors. Decorative, aesthetic engravings and the inclusion of gems to accentuate the ring are contemplated by the design.

To change out the outer shell 12, the user simply slides it and/or pulls it to detach it from the rails 34, 36 of the outer shell 14 that define the groove 16. The user needs only to clip the outer shell 12 back in place.

The PCB rigid-flex structure is designed with components restricted such as RF antenna 44, button and, fitted inside of the polymer mold or overmold housing 28 in order to have a solid and compact structure. RF antenna is placed outside the ring and near the button for optimal range of data transmission. The button position is near the electronics which communicate with the user's electronic device but is not a control for external devices. Further, the device does not diagnose any conditions based on the data collected, but merely collects it and reports it to the user's external devices to do with as the user sees fit. The outer shell renders the ring totally waterproof even with a button and charging pins. The final assembly has a very specific direction and positioning design which is made for ease of use and better accuracy when worn, i.e., on the finger 28, the flat part 22 needs to be on the bottom of the finger 28 for better data accuracy and the button 34 needs to be turned towards the wearer's thumb 38 for easy clicking of the button 34 from any finger 28. This precise positioning (bottom of the finger, on the palm side) exploits the fact that the nervous system of the finger is much more sensitive than the one of the wrist which allows a better accuracy of the optical sensor and a better sensitivity to the vibrations of the ring compared to prior art devices that are wrapped around wrist. The sensors are placed in such a way that with the correct positioning of the ring, as described herein, the user is able to gather better data. Each finger has two proper digital arteries that run on either side along its length.

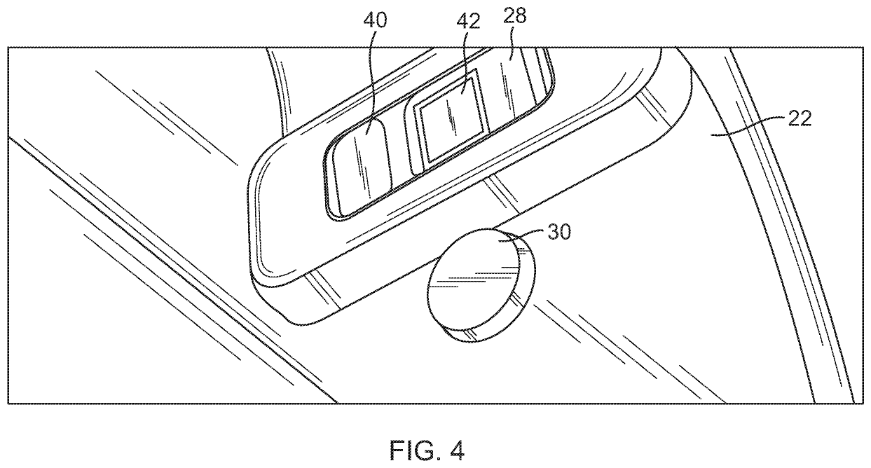

The flat portion 22 houses an infrared optical pulse sensor 40 (See FIG. 4), charging pins 30 and a vibration motor 40. The ring is 8 mm wide 2.8 mm thick and weighs only 4 grams making it one of the most discreet, seamless and lightweight wellness wearable technology devices.

The sensors allow for 24/7 tracking by recognizing specific gestures prior to activation, at all times, i.e., there is continuous wellness tracking for better recommendations by the software in the app housed in the mobile device 44. The software with the associated app allows for the calculation of various metrics, including, but not limited to: heart rate, heart rate variability, blood oxygenation levels (SpO2), blood pressure, stress detection, glucose monitoring, metabolic changes, mood and emotion tracking, seizure and fall detection, steps taken, walking equivalency, kcal burned, active minutes, VO2 max, heart rate max, recovery levels, resting Heart rate, sleep balance, activity volume, activity intensity, sleep disturbances, real sleep, circadian rhythm, REM sleep, deep sleep, light sleep, sleep latency, sleep debt, hours slept. Furthermore, the software in the associated app can provide a calculation of unique scoring systems

Precise algorithms are established based on the wearer's baseline, which is an innovative way of treating biometrics from sensors for a wearable device. Such algorithms allow the software to make recommendations to users in a unique and personal way through the establishment of the baselines for each metric mentioned above, for each user.

Other features of the device and associated app include an alarm clock that utilizes data transmitted from the ring to the user's phone in order to track above metrics and can provide a wake up in order to combat sleep inertia (“smart alarm”) (and/or manual alarm), or provide a wake ups that are in alignment with furthering wellness based on the data previously gathered. The ring automatically transmits data throughout the user's core sleep or any nap episode to calculate above metrics. The ring and associated app can provide reminders in the form of a ring vibration of a predefined event. The cross correlation of both day and night data gathering is unique to the product of the instant invention as it can be worn during both times.

The accuracy achieved by the 24/7 wearing of the instant invention is important in that it allows for more accurate and useful wellness recommendations. The performance result closely approximates laboratory level data treatment in existence for cardiac, sleep and activity science, which currently only exist in labs, which is prohibitively expensive for routine wellness as well as being very invasive.

The embedded vibration function can provide a medication reminder and can also provide a warning when the user gets too far from his phone. Additionally, when the ring is not connected, it vibrates. A curated list of activity and sleep programs take advantage of both the heart photoplethysmography sensor and the embedded accelerometer so the user is reminded of every step to take during the program and whereas the sensors continuously transmit data to the user's phone.

Related to health, an arrhythmia detection algorithm can be programmed, taking full advantage of the heart photoplethysmography sensor, whereas data is transmitted at very high frequencies to detect and prevent abnormal heart beat signatures. A tension measurement feature can be incorporated with similar principals. Likewise, a driving drowsiness detection feature can alert a drowsy driver. A sleep apnea detection algorithm can take full advantage of the heart photoplethysmography sensor and of the oxygenation levels datasets, whereas data is transmitted at specific frequencies to detect and prevent abnormal respiratory signatures.

The ring can include an emergency feature, whereas the user presses the ring's button (once or several times defined in the app), it transmits data to the user's phone and cellular network to alert any other individual of an abnormal situation. This feature is customizable as a control feature wherein when the user presses the ring's button, it triggers a specific interaction determined beforehand, linking the ring through the user's phone to other smart objects and/any connected device. A gesture enabled feature can be incorporated wherein when a specific motion of the user's finger(s) and or hand occurs, an action is triggered.

The invention of the instant invention can, by design, also house technologies, including; wireless charging, RF energy harvesting technology, near field communication, contactless payment, microphone integration, speaker integration.

The button, made of polymer is unique to this invention as the button placement and function is not found in the prior art. The outer shell, made of polymer is made and directly assembled with a 2K insert-molding process, a step not found in the prior art. This creates an indivisible outer part of the final ring that has no cavity and no flange, which is present in the prior art. The PCB Assembly (PCBA) passes through a topicalization coating process to ensure an optimal protection of an electronic circuit in a harsh or constrained environment, a step now found in the prior art. The rigid-flex PCB is then soldered to the battery which is distinguishable from prior art products that have a full flex PCB. Once it is powered by the battery, firmware is flashed to the PCBA by connecting through contact pins located on the edge of PCB. Once flashed, the edge part of the PCB that has the pins is cut. The electronics are then attached on the inside face of the outer part of the ring using specific external tooling and guides in the outer part to ensure the electronics part is aligned in the proper position. For example, the PPG sensor should be aligned with the button and the vibration motor, a step not found in prior art processes that lack a vibration motor. Then the outer part and electronics are deposited in a mold in which injections of liquid polymer are introduced to create the inner part of the final ring in contact with the finger. The mold is filled with liquid polymer to cover the unfilled space that still remains in the mold. After drying, the liquid polymer totally encompasses the electronics except the ppg sensor and the charging pins that are flush and touch the finger skin. In prior art devices, the inner ring encompasses all electronics and nothing comes out of the polymer to be in contact with the finger. The liquid polymer sticks to the outer part to form a single indivisible body part with all the described parts above. The changeable outer-shell is then added on the body part.

The interior of the ring, which is a molded body part is not an integral loop circle but has flat part and sensors part that make it not round. The flat part is made so that the ring cannot turn around the finger and that the ppg sensor always remain at the same place on the finger which is a special place that has been decided to have the best signal quality. This is unique and not found in the prior art.

The invention illustratively disclosed herein suitably may be practiced in the absence of any element which is not specifically disclosed herein.

The discussion included in this patent is intended to serve as a basic description. The reader should be aware that the specific discussion may not explicitly describe all embodiments possible and alternatives are implicit. Also, this discussion may not fully explain the generic nature of the invention and may not explicitly show how each feature or element can actually be representative or equivalent elements. Again, these are implicitly included in this disclosure. Where the invention is described in device-oriented terminology, each element of the device implicitly performs a function. It should also be understood that a variety of changes may be made without departing from the essence of the invention. Such changes are also implicitly included in the description. These changes still fall within the scope of this invention.

Further, each of the various elements of the invention and claims may also be achieved in a variety of manners. This disclosure should be understood to encompass each such variation, be it a variation of any apparatus embodiment, a method embodiment, or even merely a variation of any element of these. Particularly, it should be understood that as the disclosure relates to elements of the invention, the words for each element may be expressed by equivalent apparatus terms even if only the function or result is the same. Such equivalent, broader, or even more generic terms should be considered to be encompassed in the description of each element or action. Such terms can be substituted where desired to make explicit the implicitly broad coverage to which this invention is entitled. It should be understood that all actions may be expressed as a means for taking that action or as an element which causes that action. Similarly, each physical element disclosed should be understood to encompass a disclosure of the action which that physical element facilitates. Such changes and alternative terms are to be understood to be explicitly included in the description.

What is claimed is:

1. A wearable, wellness-tracking ring comprising:
a main body portion without a cavity of a fixed size made of a nonconductive rigid material wherein said main body portion has an outer surface, said outer surface being circular in shape and wherein said outer surface of said main body portion contains a central groove created by a first circular flange and a second circular flange, surrounding a perimeter of said outer surface of said main body portion on either side of only said outer surface;
an inner surface of said main body portion opposing said outer surface wherein an aperture is created therethrough that is only partially circular in shape through which a finger of a user can fit therebetween and wherein said inner surface further comprises
a raised portion that creates a non-circular profile for said aperture wherein said raised portion creates a flat plateau_with smooth edges that is covered by a protective top and is made of a same material as said inner surface and containing no contact sensors wherein said protective top covers one or more housed electronic components that are programmed and protected thereunder to capture and communicate data from said user wherein said flat portion, when worn, is situated near the bottom of said user's finger proximate said user's palm so that said control button is oriented toward the thumb of said user for easy access while worn;
a removable decorative, circular external outer shell affixable and detachable in said groove of said outer surface said removable shell having no cavities or grooves thereon thereby providing a smooth appearance; and a control button on said outer surface that is accessible to said user while said user's finger is placed through said aperture and said flat plateau is contacting skin on said user's finger and said button is reachable by said user.

2. The ring as defined in claim 1 wherein said main body portion and said outer shell are manufactured using an over-molding process.

3. The ring as defined in claim 1 wherein said one or more_electronic components are taken from the following: rigid-flex printed circuit board (PCB), the rigid-flex printed circuit board (PCB) comprising one or more of an infrared, red and green heart photoplethysmography sensor, an multi-axis accelerometer, a temperature sensor, electrodermal activity sensor (EDA), vibration motor, a mechanical button, flash memory, an RF Transceiver IC which has near field communication capability, power management integrated circuit, a curved rechargeable battery and contact pins or wireless charging technology for charging.

4. The ring as defined in claim 1 wherein an RF transceiver IC with near field communication capability is situated on said main body portion proximate said control button.

5. The ring as defined in claim 1 wherein electronic components are assembled as follows: the button, made of polymer and outer shell, made of polymer are assembled with an over-molding process thereby creating an outer part of the ring that has no cavity; electronics are glued to the outer part of the ring, i.e., the outer shell and button with automatic tooling; liquid polymer is injected into a mold that contains the outer part and the electronics to create the inner part that is in contact with the finger to form a single indivisible part wherein the mold forms a cavity for the inner part of the ring to embody the electronic glued on the outer-shell part.

6. The ring as defined in claim 3 wherein said data that is collected is communicated to a device, such as a cellphone, a tablet, a personal computer, a cloud provider or an AI assistant and performs no execution software function.

7. The ring as defined in claim 6 wherein software is installed in said cellphone, tablet, personal computer, cloud provider or AI assistant such that said software can utilize said collected and communicated data to provide feedback to said user wherein said feedback includes one or more of the elements taken from the following: making action recommendations for better health and wellness; provide alerts through said vibration motor such as alarms, reminders or alerting said user when said ring has traveled too far from said cell phone, tablet, personal computer, cloud provider or AI assistant.

8. A method of collecting personal health data and providing continuous feedback thereof to a user comprising the steps of:
acquiring a wearable, wellness-tracking ring, said ring further comprising:
a main body portion of a fixed size made of a nonconductive rigid material wherein said main body portion has an outer surface, said outer surface being circular in shape and wherein said outer surface of said main body portion contains a central groove created by a first circular flange and a second circular flange, surrounding a perimeter of said outer surface of said main body portion on either side of said outer surface;
an inner surface of said main body portion opposing said outer surface wherein an aperture is created therethrough that is only partially circular in shape through which a finger of a user can fit therebetween and wherein said inner surface further comprises
a raised portion that creates a non-circular profile for said aperture wherein said raised portion creates a flat plateau with smooth edges that is covered by a protective top and is made of a same material as said inner surface and containing no contact sensors wherein said protective top covers one or more housed electronic components that are programmed and protected thereunder to capture and communicate data from said user wherein said flat portion, when worn, is situated near the bottom of said user's finger proximate said user's palm so that said control button is oriented toward the thumb of said user for easy access while worn;
a removable decorative, circular external outer shell affixable and detachable in said groove of said outer surface said removable shell having no cavities or grooves thereon thereby providing a smooth appearance; and a control button on said outer surface that is accessible to said user while said user's finger is placed through said aperture and said flat plateau is contacting skin on said user's finger and said button is reachable by said user;
inserting a user's finger into said aperture;
pairing said ring to said user's personal external computing device;
wearing said ring continuously such that continuous data is communicating said external computing device; and
collecting said continuous data into said external computing device for use by said user.

9. The method as defined in claim 8 wherein said main body portion and said outer shell are manufactured using an over-molding process.

10. The method as defined in claim 8 wherein said one or more_electronic components are taken from the following: rigid-flex printed circuit board (PCB), the rigid-flex printed circuit board (PCB) comprising one or more of an infrared, red and green heart photoplethysmography sensor, an multi-axis accelerometer, a temperature sensor, electrodermal activity sensor (EDA), vibration motor, a mechanical button, flash memory, an RF Transceiver IC which has near field communication capability, power management integrated circuit, a curved rechargeable battery and contact pins or wireless charging technology for charging.

11. The method as defined in claim 8 wherein an RF transceiver IC with near field communication capability is situated on said main body portion proximate said control button.

12. The method as defined in claim 8 wherein electronic components are assembled as follows: the button, made of polymer and outer shell, made of polymer are assembled with an over-molding process thereby creating an outer part of the ring that has no cavity; electronics are glued to the outer part of the ring, i.e., the outer shell and button with automatic tooling; liquid polymer is injected into a mold that contains the outer part and the electronics to create the inner part that is in contact with the finger to form a single indivisible part wherein the mold forms a cavity for the inner part of the ring to embody the electronic glued on the outer-shell part.

13. The method as defined in claim 8 wherein said data that is collected is communicated to a device, such as a cellphone, a tablet, a personal computer or an AI assistant and performs no execution software function.

14. The method as defined in claim 13 wherein software is installed in said cellphone, tablet, personal computer or AI assistant such that said software can utilize said collected and communicated data to provide feedback to said user wherein said feedback includes one or more of the elements taken from the following: making action recommendations for better health and wellness; provide alerts through said vibration motor such as alarms, reminders or alerting said user when said ring has traveled too far from said cell phone, tablet, personal computer or AI assistant.

15. The method as defined in claim 8 wherein said control button is utilized as an emergency signaler to said cell phone, tablet, personal computer or AI assistant.

16. The method as defined in claim 8 wherein said ring and associated software are programmed to induce sleep, give indications, interact with the user and evoke relaxation through pace breathing and haptic feedback.
